(19) United States
(12) Patent Application Publication
(10) Pub. No.: US 2012/0316338 A1
(43) Pub. Date: Dec. 13, 2012

(54) Methods and Intermediates for the Preparation of Optionally Radio-Labeled Imatinib

(76) Inventors: Rhys Salter, Basel (CH); Maria Ines Rodriguez Perez, Biel-Benken (CH); Thomas Moenius, Inzlingen (DE); Rolf Voges, Lorrach (DE); Hendrik Andres, Hochwald (CH); Kirk Bordeaux, Flanders, NJ (US)

(21) Appl. No.: 13/526,115

(22) Filed: Jun. 18, 2012

Related U.S. Application Data
(63) Continuation of application No. 11/917,353, filed on Dec. 13, 2007.

(30) Foreign Application Priority Data
Jun. 14, 2005 (GB) 0512091.0

(51) Int. Cl. C07D 401/14

(52) U.S. Cl. 544/295

(57) ABSTRACT

The invention relates to new processes for the manufacture of N-{5-[4-(4-methyl-piperazino-methyl)-benzoylamido]-2-methylphenyl}-4-(3-pyridyl)-2-pyrimidine-amine (compound of formula I,

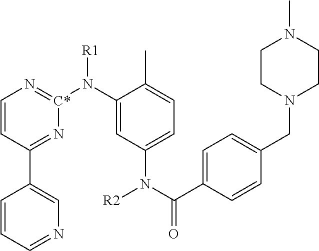

new processes for the manufacture of metabolites of N-{5[4-(4-methyl-piperazino-methyl)-benzoylamido]-2-methylphenyl}-4-(3-pyridyl)-2-pyrimidine-amine observed after administration of the compound to warm-blooded animals as well as to intermediates used in said processes. New starting materials as well as processes for the preparation thereof are likewise the subject of this invention. The processes described herein are especially suitable to furnish said compounds having isotopic labeling. The such obtained labeled compounds are in particular suitable to track and to investigate into the metabolism of N-{5-[4-(4-methyl-piperazino-methyl)-benzoylamido]-2-methylphenyl}-4-(3-pyridyl)-2-pyrimidine-amine and its pharmaceutically acceptable salts in clinical and pre-clinical studies.

The mesylate salt of N-{5-[4-(4-methyl-piperazino-methyl)-benzoylamido]-2-methylphenyl}-4-(3-pyridyl)-2-pyrimidine-amine (compound of formula I, Imatinib) is marketed under the brand Glivec® (Gleevec®). Glivec® is a tyrosine kinase inhibitor suitable for the treatment of chronic myeloid leukemia and GIST (gastro-intestinal stromal tumors). N-{5-[4-(4-methyl-piperazino-methyl)-benzoylamido]-2-methylphenyl}-4-(3-pyridyl)-2-pyrimidine-amine can be prepared, e.g. as disclosed in U.S. Pat. No. 5,521,184. The monomesylate salt of N-{5-[4-(4-methyl-piperazino-methyl)-benzoylamido]-2-methylphenyl}-4-(3-pyridyl)-2-pyrimidine-amine can be prepared and formulated, e.g., as described in Examples 4 and 6 of WO 99/03854 or as described in WO03/090720.

The invention relates to new processes for the manufacture of N-{5-[4-(4-methyl-piperazino methyl)-benzoylamido]-2-methylphenyl}-4-(3-pyridyl)-2-pyrimidine-amine (compound of formula I, Imatinib), new processes for the manufacture of metabolites of N-{5-[4-(4-methyl-piperazino-methyl)-benzoylamido]-2-methylphenyl}-4-(3-pyridyl)-2-pyrimidine-amine observed after administration of the compound to warm-blooded animals as well as to intermediates used in said processes. New starting materials as well as processes for the preparation thereof are likewise the subject of this invention. The processes described herein are especially suitable to furnish said compounds having isotopic labeling. The such obtained labeled compounds are in particular suitable to track and to investigate into the metabolism of N-{5-[4-(4-methyl-piperazino-methyl)-benzoylamido]-2-methylphenyl}-4-(3-pyridyl)-2-pyrimidine-amine and its pharmaceutically acceptable salts in clinical and preclinical studies.

Based on the disclosure of the present patent specification, a person skilled in the art is capable to prepare N-oxide derivatives of the compounds described herein beyond those N-oxides specifically mentioned, pro-drug derivatives, protected derivatives, individual isomers and mixture of isomers thereof, as well as pharmaceutically acceptable salts thereof.

In a first embodiment, the mesylate salt of a compound of formula I

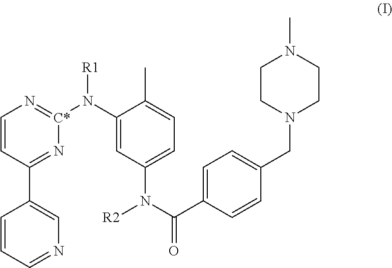

wherein R, and R₂ are both hydrogen and C* denotes carbon having the natural distribution of isotopes or, alternatively, being labeled by enrichment of one carbon isotope, e.g. ¹⁴C, is prepared by reacting 1-methylpiperazine with a compound of formula II,

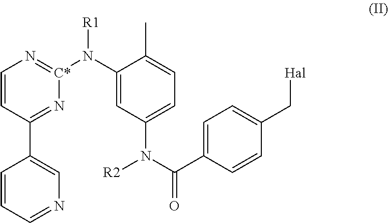

wherein Hal represents halogen, preferably chloro, and the other radicals and symbols have the meanings as defined above for a compound of formula I.

A compound of formula II wherein Hal represents halogen, preferably chloro, and the other radicals and symbols have the meanings as defined above for a compound of formula I can be obtained by first reacting the hydrochloric acid addition salt of formula III,

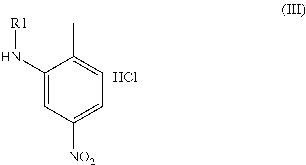

wherein R₁ has the meaning as defined above for a compound of formula I, with H₂NC*N, wherein C* denotes carbon having the natural distribution of isotopes or, alternatively, being labeled by enrichment of one carbon isotope, e.g. ¹⁴C, furnishing the guanidinyl substituted nitrophenyl derivative of formula IV

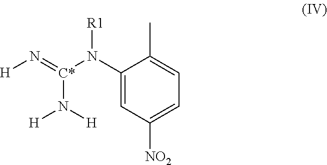

wherein the radicals and symbols have the meanings as defined above for a compound of formula I. In a second step the guanidinyl substituted nitro phenyl derivative of formula IV is further reduced to the corresponding guanidinyl substituted amino phenyl derivative of formula V

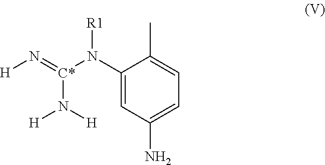

wherein the radicals and symbols have the meanings as defined above for a compound of formula I. By reacting such guanidinyl substituted amino phenyl derivative of formula V in a third step with the pyridyl derivative of formula VI,

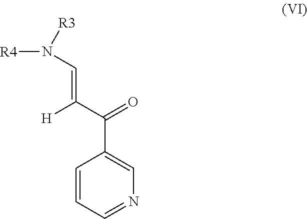

wherein R₃ and R₄ are both C₁₋₄alkyl, the pyridyl pyrimidine of formula VII,

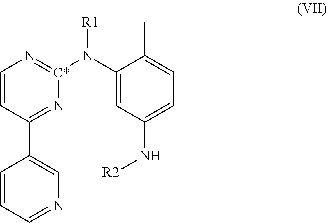

wherein the radicals and symbols have the meanings as defined above for a compound of formula I, is obtained. Such pyridyl pyrimidine of formula VII is finally reacted with 4-halomethyl-benzoic acid to furnish the amide of formula II.

The pyridyl pyrimidine of formula VII wherein the radicals and symbols have the meanings as defined above for a compound of formula I can also be prepared by the following route. In a first step, the compound of formula I, wherein the radicals and symbols have the meanings as defined above, is reacted with a reagent replacing the hydrogen atoms attached to nitrogen atoms by protecting groups, thus furnishing a compound of formula I

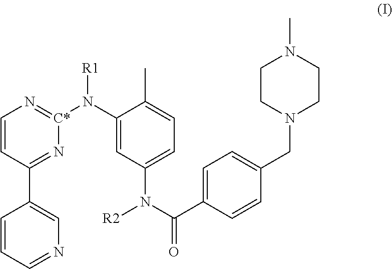

wherein R₁ and R₂ both represent protecting groups and C* denotes carbon having the natural distribution of isotopes or, alternatively, being labeled by enrichment of one carbon isotope, e.g. ¹⁴C. In a second step the benzoic acid unit of the molecule is removed by hydrolysis resulting in the free amine of formula XII,

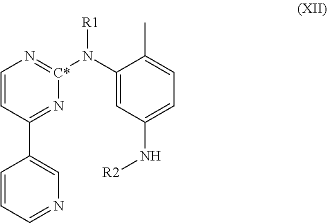

wherein R₁ and R₂ both represent protecting groups and C* denotes carbon having the natural distribution of isotopes or, alternatively, being labeled by enrichment of one carbon isotope, e.g. ¹⁴C, followed by replacing the protecting groups R₁ and R₂ by hydrogen, i.e. the removal of the protecting groups, delivering a compound of formula II, wherein the radicals and symbols have the meanings as defined above for a compound of formula I.

In a second embodiment, compound of formula VIII

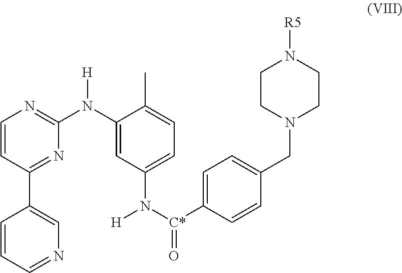

wherein R₅ is hydrogen or C₁₋₄alkyl and C* denotes carbon having the natural distribution of isotopes or, alternatively, being labeled by enrichment of one carbon isotope, e.g. ¹⁴C, can be obtained by reaction of a compound of formula X,

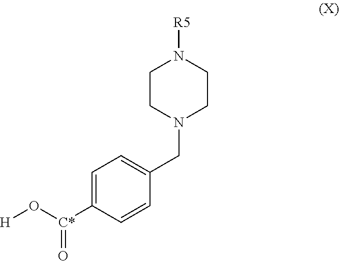

wherein R₅ is a protecting group, e.g. tert-butoxycarbonyl, or C₁₋₄alkyl and C* denotes carbon having the natural distribution of isotopes or, alternatively, being labeled by enrichment of one carbon isotope, e.g. ¹⁴C, with a compound of formula XI,

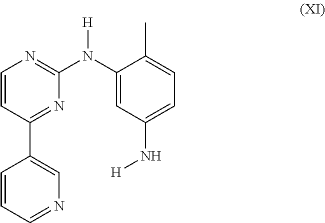

followed by splitting of the protecting group R₅, in case R₅ constitutes a protecting group.

The compound of formula X,

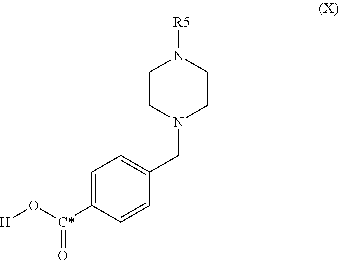

wherein the radicals and symbols have the meanings as defined above can be obtained by reacting a compound of formula IX

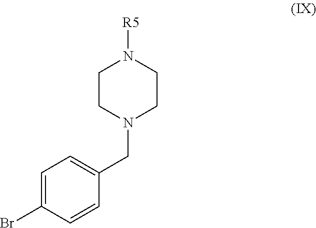

wherein R₅ is a protecting group, e.g. tert-butoxycarbonyl, or C₁₋₄alkyl first with t-butyl lithium to prepare the corresponding lithio derivative and to react such lithio derivative with C*O₂, wherein C* denotes carbon having the natural distribution of isotopes or, alternatively, being labeled by enrichment of one carbon isotope, e.g. ¹⁴C.

In a further aspect, the present invention relates to the preparation of a compound of formula XIII

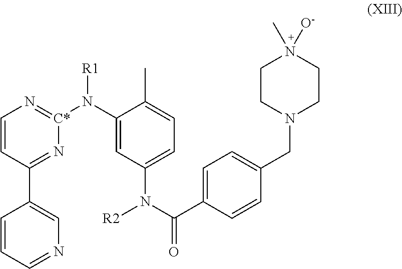

wherein the radicals and symbols have the meanings as defined above for a compound of formula I, by oxidation of a compound of formula I, wherein the radicals and symbols have the meanings as defined above.

In an additional aspect, the present invention relates to the preparation of a compound of formula XIV

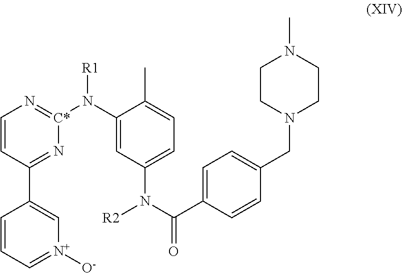

wherein the radicals and symbols have the meanings as defined above for a compound of formula I, by reacting a compound of formula XVI,

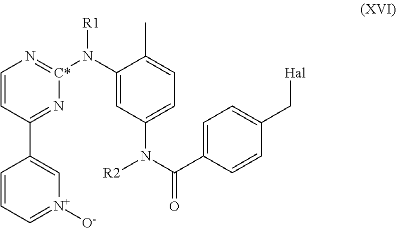

wherein Hal represents halogen, preferably chloro, and the other radicals and symbols have the meanings as defined above for a compound of formula I with 1-methylpiperazine.

The starting material, the compound of formula XVI, wherein Hal represents halogen and the other radicals and symbols have the meanings as defined above for a compound of formula I can be obtained by the following process. Firstly, the free amine of formula VII, wherein R₁ and R₂ are both hydrogen, is reacted with an agent introducing a protecting group under conditions effecting selective replacement of one hydrogen atom of a primary amine in the presence of a secondary amino group, followed by oxidation of the pyridyl nitrogen atom, e.g. with MCPBA, furnishing the N-oxide of formula XV,

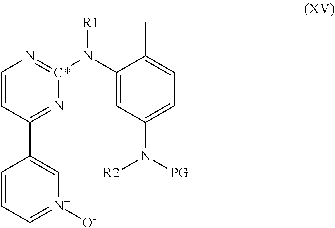

wherein R₁ and R₂ are both hydrogen, PG denotes a protecting group and the other symbols have the meanings as defined above for a compound of formula I. Such N-oxide of formula XV is first subjected to conditions removing the protecting group PG and the obtained free amine thereafter reacted with 4-halomethyl benzoic acid furnishing the compound of formula XVI, wherein Hal represents halogen and the other radicals and symbols have the meanings as defined above for a compound of formula I

In a further embodiment, the present invention provides a compound of formula XVIII

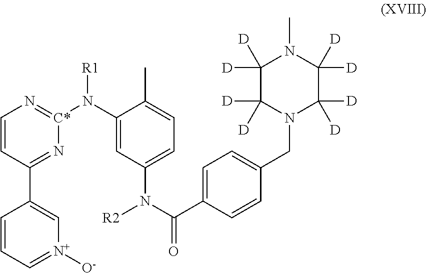

wherein the radicals and symbols have the meanings as defined above for a compound of formula I, by reacting a compound of formula II,

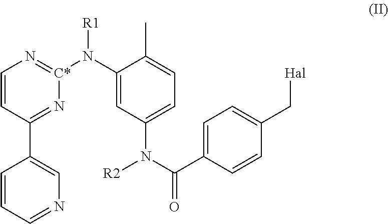

wherein Hal represents halogen, preferably chloro, and the other radicals and symbols have the meanings as defined above for a compound of formula I, with the piperazine derivative of formula XVII.

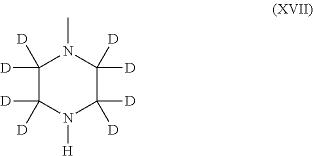

Finally, the present invention relates to the preparation of a compound of formula XX

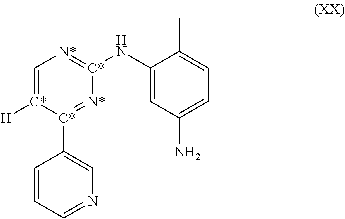

wherein C* denotes carbon having the natural distribution of isotopes or, alternatively, being labeled by enrichment of one carbon isotope, e.g. ¹³C, and N* denotes nitrogen having the natural distribution of isotopes or, alternatively, being labeled by enrichment of one nitrogen isotope, e.g. ¹⁵N, comprising reacting the pyridine derivative of formula XXI

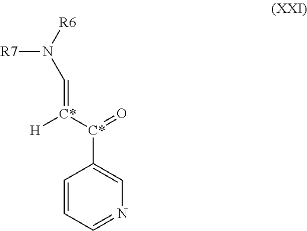

wherein R₆ and R₇ are both C₁₋₄alkyl and the other symbols have the meanings as defined above for a compound of formula XX, with the phenyl guanidine derivative of formula XXII,

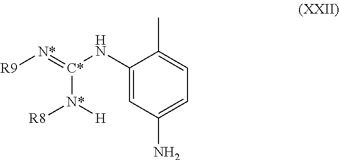

wherein R₈ and R₉ are both hydrogen and the other symbols have the meanings as defined above for a compound of formula XX.

The pyridine derivative of formula XXI wherein R₆ and R₇ are both C₁₋₄alkyl and the other symbols have the meanings as defined above for a compound of formula XX, can be prepared starting with 3-trimethylstannyl pyridine and reacting such compound with the acetyl halide of formula XXIV,

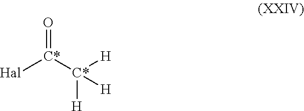

wherein Hal denotes halo and the other symbols have the meanings as defined above for a compound of formula XX, resulting in the acetyl pyridine of formula XXIII

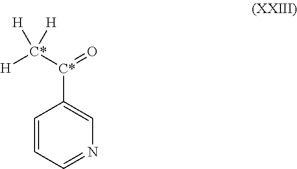

wherein the symbols have the meanings as defined above for a compound of formula XX. Such acetyl pyridine of formula XXIII is further reacted with di-C₁₋₄alkyl formamide di-C₁₋₄alkyl acetal, resulting in the desired pyridine derivative of formula XXI wherein R₆ and R₇ are both C₁₋₄alkyl and the other symbols have the meanings as defined above for a compound of formula XX.

The amino phenyl guanidine derivative of formula XXII, wherein R₈ and R₉ are both hydrogen and the other symbols have the meanings as defined above for a compound of formula XX, can be prepared starting with the thiourea derivative of formula XXV,

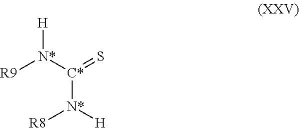

wherein R₈ and R₉ are both hydrogen and the other symbols have the meanings as defined above for a compound of formula XX, and reacting such thiourea derivative of formula XXV with a reagent exchanging one hydrogen atom of each amino group by a protecting group, e.g. tert-butyloxy carbonyl, resulting in a thiourea derivative of formula XXV, wherein R₈ and R₉ both represent a protecting group. Such protected thiourea derivative of formula XXV is then reacted with 2-methyl-5-nitroaniline to furnish the nitro phenyl guanidine derivative of formula XXVI,

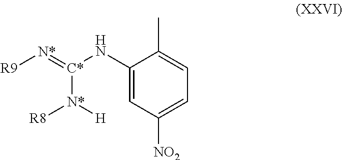

wherein R₈ and R₉ both represent a protecting group and the other symbols have the meanings as defined above for a compound of formula XX. Splitting off both protecting groups followed by reduction of the nitro function, results finally in the amino phenyl guanidine derivative of formula XXII wherein R₈ and R₉ are both hydrogen and the other symbols have the meanings as defined above for a compound of formula XX.

All remaining starting materials of are known, capable of being prepared according to known processes, or commercially obtainable; in particular, they can be prepared using processes as described in the Examples.

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralizing agents, for example ion exchangers, typically cation exchangers, for example in the H+ form, depending on the type of reaction and/or reactants at reduced, normal, or elevated temperature, for example in the range from −100° C. to about 190° C., preferably from about −80° C. to about 150° C., for example at −80 to −60° C., at room temperature, at −20 to 40° C. or at the boiling point of the solvent used, under atmospheric pressure or in a closed vessel, where appropriate under pressure, and/or in an inert atmosphere, for example under argon or nitrogen. The solvents from which those can be selected which are suitable for the reaction in question include for example water, esters, typically lower alkyl-lower alkanoates, e.g diethyl acetate, ethers, typically aliphatic ethers, e.g. diethylether, or cyclic ethers, e.g. tetrahydrofuran, liquid aromatic hydrocarbons, typically benzene or toluene, alcohols, typically methanol, ethanol or 1- or 2-propanol, nitriles, typically acetonitrile, halogenated hydrocarbons, typically dichloromethane, acid amides, typically dimethylformamide, bases, typically heterocyclic nitrogen bases, e.g. pyridine, carboxylic acids, typically lower alkanecarboxylic acids, e.g. acetic acid, carboxylic acid anhydrides, typically lower alkane acid anhydrides, e.g. acetic anhydride, cyclic, linear, or branched hydrocarbons, typically cyclohexane, hexane, or isopentane, or mixtures of these solvents, e.g. aqueous solutions, unless otherwise stated in the description of the process. Such solvent mixtures may also be used in processing, for example through chromatography or distribution.

The compounds of the invention can be prepared as pharmaceutically acceptable acid addition salts by reacting the free base form of the compounds with pharmaceutically acceptable inorganic or organic acids. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Pro-drug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate pro-drugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

A detailed example of the synthesis of the compound described above can be found in the Examples. In the preferred embodiment, the compounds described above are prepared according to or in analogy to the processes and process steps defined in the Examples.

### EXAMPLES

The present invention is further exemplified, but not limited, by the following Examples that illustrate the preparation of compounds described above.

### ABBREVIATIONS

DMAP dimethyl aminopyridine

DMSO dimethyl sulfoxide

MCPBA m-chloroperbenzoic acid

MeOH methanol

NMR nuclear magnetic resonance

RT room temperature

THF tetrahydrofuran

TLC thin layer chromatography

w/v weight per volume

### Example 1

### 4-(4-methyl-piperazin-1-ylmethyl)-N-[4-methyl-3-(4-pyridin-3-yl-[2-¹⁴C]pyrimidin-2ylamino)-phenyl]benzamide mesylate

1-Methylpiperazine (3 ml, 27 mmol) is added to a solution of the hydrochloride salt of 4-chloromethyl-N-{4-methyl-3-[4-(1-oxy-pyridin-3-yl)-[2-¹⁴C]pyrimidin-2-ylamino]-phenyl}-benzamide (step 1.5, 335 mg, 0.71 mmol) in ethanol (20 ml) and the resulting mixture is stirred for 5 h at 45° C. The suspension is cooled to RT and water (4.4 ml) followed by 20% w/v sodium hydroxide (130 μl) is added. The resulting suspension is stirred for 30 minutes at 90° C., then cooled to RT, centrifuged and the aqueous solvent removed. The beige crystals are washed further 2 times with water and dried under high vacuum. Finally the solid is purified flash chromatography on silica gel, eluting with dichloromethane : MeOH 90:10 and 1% ammonia to afford the intermediate as a yellow oil. Finally the obtained intermediate is dissolved in MeOH (4 ml), methane sulphonic acid is added (21 μl) and the obtained solutions is stirred. The obtained product is crystallized out of solution by seeding with inactive reference product to give the title product of specific activity 1.73 GBq/mmol. ¹H-NMR 400 MHz (DMSO-d₆) δ: 2.21 (s, 3H); 2.3 (s, 3H); 2.8 (s, 3H); 2.85-3.1, m, 4H); 3.25-3.4 (m, 4H); 3.62 (br.s, 3H); 7.18 (d, J=8Hz; 1H); 7.38-7.51 (m, 4H); 7.91 (d, 7.6 Hz, 1H); 8.5 (d, J=1.4 Hz, 1H); 8.45 (d, J=7.8, 1H); 8.49 (d, J =5 Hz, 1H); 8.65 (dd, J=1.5 and 4.6 Hz, 1H); 8.95 (s, 1H); 9.22 (d, J=1.5 Hz, 1h); 9.29 (br.s, 1H), 10.12 (s, 1H); MS: (MH+, 496.4)

Step 1.1: [¹⁴C]Cyanamide

Barium [¹⁴C]carbonate (1.18 g, 6 mmol, 10.4 GBq, 1.79 GBq/mmol) is placed in a quartz glass cylindrical tube and heated to 800° C. while a gentle stream of ammonia gas is passed through. After 3 hours, the crude barium [¹⁴C]cyanamide product is allowed to cool to RT and the stream of ammonia ceased. The crude barium[¹⁴C]cyanamide (1.063 g, 6 mmol) is suspended in water (10 ml) and dissolved by ultrasonication. 2M Sulphuric acid (2.6 ml) is added carefully dropwise with stirring until pH 5.5. The water is removed at 60° C. at 23 Torr. The residue is cooled with dry ice/acetone and the residual water lyophilised to give a crystalline solid product. This is taken up into diethyl ether and filtered to remove insoluble residue. The ether is removed and the obtained residue dissolved in t-butanol.

Step 1.2: N-(2-methyl-5-nitro-phenyl)-[¹⁴C]quanidine

To a stirred solution of 2-methyl-4-nitroaniline (1.52 g, 19 mmol) in t-butanol at 40° C. is added 4M hydochloric acid in dioxane (5 ml) to give a yellow suspension. The solvent is removed and the dry residue added to a solution of crude [¹⁴C]cyanamide (step 1.2) in t-butanol (5 ml) to give a yellow suspension. After heating to 100° C. for 1 hour, a solution is obtained. After a further 5 hours, the solvent is removed at 45° C. and the residue treated with 3% w/v sodium hydroxide (10 ml) and extracted with dichloromethane (3×30 ml). The organic phases are combined and evaporated and chromatographed on silica gel eluting with 75:25:0.5:0.5 dichloromethane:MeOH:water:acetic acid to give the title product, 6.07 GBq.

Step 1.3: N-(5-amino-2-methyl-phenyl)-[¹⁴C]quanidine

A mixture of N-(2-methyl-5-nitro-phenyl)[¹⁴C]guanidine (step 1.2) (760 mg, 3.91 mmol) in n-butanol (25 ml) containing 10% Pd/C (125 mg) is stirred under 1 atmosphere of hydrogen gas at RT for 4.5 hours. The mixture is filtered through Hyflo (Celite), washed with a further portion of n-butanol (3×10 ml), combined and the n-butanol removed on a rotary evaporator at 45° C. to give the crude product as a colourless oil. Radio TLC 80:25:0.5:0.5 dichloro-methane:MeOH:water:acetic acid, on silica gel F254 plates RF=0.34; Total activity 3.7 GBq.

Step 1.4: 4-methyl-N*3*-(4-pyridin-3-yl-[2-¹⁴C]pyrimidin-2-yl)-benzene-1,3-diamine

A solution of crude N-(5-amino-2-methyl-phenyl)-[¹⁴C]guanidine (step 1.4, 476 mg, 2.89 mmol) and 3-dimethylamino-1-pyridin-3-yl-propenone (509 mg, 2.89 mmol) in n-butanol (30 ml) is heated at reflux (130° C.) for 3.5 hours. Removal of the solvent and purification by flash chromatography eluting with 2:98 pentane:acetone gives the title product as a yellow foam. Radio TLC, 2:98 pentane:acetone, on silica gel F₂₅₄ plates RF=0.5. Total activity, 2.3 GBq.

Step 1.5: 4-chloromethyl-N-[4-methyl-3-(4-pyridin-3-yl-[2-¹⁴C]Pyrimidin-2-ylamino)-phenyl]-benzamide

To a solution of 4-methyl-N*3*-(4-pyridin-3-yl-[2-¹⁴C]pyrimidin-2-yl)-benzene-1,3-diamine (step 1.4, 655mg, 2.36 mmol) in dry THF (20 ml) under argon at 0° C. is added dropwise a solution of 4-(chloromethyl)-benzoyl chloride (2.646 g, 14 mmol) in THF (10 ml) and the corresponding mixture is stirred at RT for 16 h. The resulting suspension is centrifuged, the precipitate product is washed with ether, re-centrifuged, the ether removed and the solid dried to afford the hydrochloride salt of the title product.

### Example 2

### 4-(piperazin-1-ylmethyl)-N-[4-methyl-3-(4-pyridin-3-yl-[2-¹⁴C]pyrimidin-2-ylamino)-phenyl]-benzamide hydrochloride

To 4-{4-[4-methyl-3-(4-pyridin-3-yl-pyrimidin-2-ylamino)-phenyl[¹⁴C]carbamoylybenzyl}-piperazine-1-carboxylic acid tert-butyl ester (step 2.3, 200.5 mg, 0.536 mmol, 486.8 MBq) is added 3M HCl in MeOH (20 ml). After 30 minutes, an additional 10 ml of 3M HCl in MeOH is added and the reaction stirred for a further 60 minutes. The product is filtered off under suction through a glass frit and washed with first cold MeOH (5 ml) followed by ethyl acetate (100 ml) and allowed to dry under high vacuum to give the title compound as orange crystals.

Step 2.1: 4-(4-Bromo-benzyl)-piperazine-1-carboxylic acid tert-butyl ester

A mixture of N-boc-piperazine (4.94 g, 26.5 mmol), p-bromo-benzylbromide (5.52 g, 22 mmol) and K₂CO₃ (6.4 g, 46 mmol) in MeOH (50 ml) is stirred for 90 minutes at RT. The mixture is filtered through a glass frit and the solvent evaporated. The residue is taken up into dichloromethane, filtered a second time though a glass frit and washed with dichloromethane. The filtrate is evaporated and the crude colourless oil chromatographed on silica gel eluting with diethyl ether/n-hexane (10:90 to 50:50) to give the title product as a white solid.

Step 2.2: 4-(4-[¹⁴C]Carboxy-benzyl)-piperazine-1-carboxylic acid tert-butyl ester

A solution of t-butyl lithium 1.5 M in hexane (0.97 ml, 1.46 mmol) is added at −78° C. to a solution of 4-(4-bromo-benzyl)-piperazine-1-carboxylic acid tert-butyl ester (step 2.1, 285 mg, 0.8 mmol) in anhydrous THF (5 ml) and the mixture is stirred at −78° C. for 4 minutes. The resulting lithio derivative is cooled to −192° C., allowed to react with [¹⁴C]carbon dioxide generated by addition of concentrated sulphuric acid to barium [¹⁴C]carbonate (0.85 eq, 122 mg, 0.62 mmol, 1378 MBq, Amersham Pharmacia Biotech) and then warmed to −78° C. and stirred for 30 minutes. The reaction is quenched with MeOH (1 ml) at −78° C., then allowed to warm to RT. The solvent is removed in vacuo and saturated ammonium chloride is added. The mixture is extracted with ethyl acetate (3×15 ml), combined and dried over sodium sulphate. Filtration and removal of the solvent gives crude white solid. The crude product is chromatographed on silica gel eluting first with diethyl ether followed by 10% MeOH 90% diethyl. The fractions are combined, evaporated on a rotary evaporater, then high vac to afford the title product as a white solid.

Step 2.3: 4-{4-[4-Methyl-3-(4-pyridin-3-yl-pyrimidin-2-ylamino)-phenyl[¹⁴C]carbamoyl]-benzyl}-piperazine-1-carboxylic acid tert-butyl ester

To a stirred solution of 4-(4-[¹⁴C]carboxy-benzyl)-piperazine-1-carboxylic acid tert-butyl ester (step 2.2, 171.5 mg, 0.536 mmol, 1114.26 MBq) in THF (5 ml) at RT is added triethylamine (332 μl, 2.4 mmol) dropwise followed by dropwise addition of iso-butyl chloroformate (78 μl, 1.0 mmol). The mixture is stirred for a further 3 minutes followed by the addition of 4-methyl-N-3-(4-pyridin-3-yl-pyrimidin-2-yl)-benzene-1,3-diamine (165 mg, 1.0 mmol). After 18 hours, the solution is evaporated and the residue subjected to flash chromatography on silica eluting first with ethyl acetate then 10% MeOH/90% ethyl acetate, then 35% MeOH/65% ethyl acetate to isolate the title product as a light yellow solid.

### Example 3

### 4-Methyl-N*3*-(4-pyridin-3-yl-[2-¹⁴C]pyrimidin-2-yl-benzene-1,3-diamine

A solution of (5-tert-Butoxycarbonylamino-2-methyl-phenyl)-(4-pyridin-3-yl-[2-¹⁴C]pyrimidin-2-yl)-carbamic acid tert-butyl ester (step 3.2, 86 mg, 0.18 mmol) and trifluoroacetic acid (463 mg, 310 μl, 4.06 mmol) is stirred over night at RT. Based on an in-process control (RTLC, silica gel, CH₂Cl₂:MeOH 95:5) no starting material is detectable. The solvent is removed in vacuo and the crude material distributed between 1N aqueous HCl and CH₂Cl₂. After re-extracting the organic phase, the acidic aqueous fractions are combined, basified with 2N aqueous NaOH and extracted three times with CH₂Cl₂. After washing the organic phase with brine, the organic phase is dried over Na₂SO₄ and the solvent evaporated. The crude material (48 mg) is purified by flash chromatography (silica gel, CH₂Cl₂:MeOH 98.5:1.5) to give the title product. Specific activity 1.934 GBq/mmol. MS: +cESI: 279.89 M+. ¹H-NMR (500.1 MHz, d⁶-DMSO): 2.08 (s, 3H), 4.88 (s, 2H), 6.35 (dd, 1H, J =2 Hz, J=7.9 Hz), 6.8 (d, 1H, J=1.7 Hz), 6.80 (d, 1H, 8.1 Hz), 7.37 (d, 1H, 5.3 Hz), 7.55 (dd, 1H, J=4.8 Hz, J=8 Hz), 8.41 (m, 1H), 8.47 (d, 1H, J=5.2 Hz), 8.67 (s, 1H), 8.70 (dd, 1H, J=1.5 Hz, J=4.9 Hz), 9.25 (d, 1H, J=1.9 Hz).

Step 3.1: (5-{tert-Butoxycarbonyl-[4-(4-methyl-piperazin-1-ylmethyl)-benzoyl]-amino}-2-methyl-phenyl)-(4-pyridin-3-yl-[2-¹⁴C]pyrimidin-2-yl)-carbamic acid tert-butyl ester

After addition of 4-dimethylaminopyridine (30 mg, 0.24 mmol) to a suspension of 4-(4-methyl-piperazin-1-ylmethyl)-N-[4-methyl-3-(4-pyridin-3-yl-[2-¹⁴C]pyrimidin-2-ylamino)-phenyl]-benzamide (Example 1, 120 mg, 0.24 mmol) and di-tent-butyl dicarbonate (265 mg, 1.22 mmol) in CH₂Cl₂ (2 ml) under Argon at RT an orange-colored solution is formed which is stirred at RT over night. The solvent is removed in vacuo and the crude product chromatographed on silica eluting with CH₂Cl₂:MeOH 93:7. The fractions are combined, evaporated on a rotary evaporator, then high vac to give the title product.

Step 3.2: (5-tert-Butoxycarbonylamino-2-methyl-phenyl)-(4-pyridin-3-yl-[2-¹⁴C]pyrimidin-2-yl)-carbamic acid tert-butyl ester

A solution of (5-{tert-butoxycarbonyl-[4-(4-methyl-piperazin-1-ylmethyl)-benzoyl]-amino}-2-methyl-phenyl)-(4-pyridin-3-yl-[2-¹⁴C]pyrimidin-2-yl)-carbamic acid tert-butyl ester (step 3.1, 153 mg, 0.22 mmol) and 2-diethylaminoethylamine (53 mg, 64 μl, 0.45 mmol) in THF (0.8 ml) is stirred over night at RT. Based on an in-process control (RTLC, silica gel, CH₂Cl₂:MeOH 95:5) additional 2-diethylaminoethylamine (159 mg, 192 μl, 1.35 mmol) is dosed until nearly no starting material is detectable. After removing the solvent in vacuo the crude product is purified by flash chromatography (silica gel, CH₂Cl₂:MeOH 98:2) to give the title product.

### Example 4

### 4-(4-Methyl-4-oxy-piperazin-1-ylmethyl)-N-[4-methyl-3-(4-pyridin-3-yl-pyrimidin-2-ylamino)-phenyl]-benzamide

A solution of 4-(4-methyl-piperazin-1-ylmethyl)-N-[4-methyl-3-(4-pyridin-3-yl-[2-¹⁴C]pyrimidin-2-ylamino)-phenyl]-benzamide (Example 1, 414 mg, 43.4 mCi, 1605.8 MBq) in anhydrous dichloromethane (10 ml) is treated with MCPBA (260 mg, 1.5 mmole) at RT for 2 h. The precipitate formed is filtered off, the filtrate evaporated and the crude product purified by flash chromatography on silica gel eluting with dichloromethane:MeOH 70:35 to afford the title product as a white solid. The product is crystallised from ethanol/ethylacetate. ¹H-NMR 400 MHz (DMSO-d₆) δ: 2.21 (s, 3H), 2.78-2.89 (m, 4H); 3.01 (s, 3H); 3.28-3.38 (m, 4H); 3.6 (s, 2H); 7.18 (d, J=8.3 Hz, 1H); 7.38-7.51 (m, 5H); 7.89 (s, 1H); 7.91 (s, 1H); 8.03 (s, 1H); 8.45 (d, J=7.9 Hz, 1H); 8.49 (d, J=5.4 Hz, 1H); 8.66 (dd, J=2, 4.2, 1H); 8.98 (s, 1H); 9.25 (d, J=2; 1H); 10.2 (s, 1H); MS: (MH+=512)

### Example 5

### N-{4-Methyl-3-[4-(1-oxy-pyridin-3-yl)-[2-¹⁴C]pyrimidin-2-ylamino]-phenyl}-4-(4-methyl-piperazin-1-ylmethyl)-benzamide

1-Methylpiperazine (2 ml, 18 mmol) is added to a solution of 4-chloromethyl-N-{4-methyl-3-[4-(1-oxy-pyridin-3-yl)-[2-¹⁴C]pyrimidin-2-ylamino]-phenyl}-benzamide (step 5.2, 151 mg, 0.34 mmole) in ethanol (20 ml) and the resulting mixture stirred for 1 h at 60° C. The solution is evaporated and the residue purified by flash chromatography on silica gel, eluting with dichloromethane:MeOH 90:10 and 1% ammonia to afford a yellow oil. The oil is crystallised from dichloromethane:MeOH to afford the N-oxide product as a crystalline solid, 175.3 MBq. ¹H-NMR 400 MHz (DMSO-d₆) δ: 2.15 (s, 3H); 2.21 (s, 3H); 2.25-2.42 (m, 4H); 3.28-3.34 (m, 4H); 3.52 (s, 2H); 7.19 (d, J=9.1 Hz; 1H); 7.38-7.42 (m, 3H); 7.45-7.52 (m, 2H); 7.88 (s, 1H); 7.89 (s, 1H); 7.99-8.2 (m, 2H); 8.28 (d, J=6.8 Hz, 1H); 8.5 (d, J=5.4 Hz, 1H); 8.8 (2, 1H); 9.2 (s, 1H), 10.1 (s, 1H).

Step 5.1: {4-Methyl-3-[4-(1-oxy-pyridin-3-yl)-[2-¹⁴C]pyrimidin-2-ylamino]-phenyl}-carbamic acid tert-butyl ester

A suspension of 4-methyl-N-3-(4-pyridin-3-yl-[2-¹⁴C]pyrimidin-2-yl)-benzene-1,3-diamine (Example 3, 286 mg, 1.03 mmole, 1890 MBq) in THF (10 ml) is treated with di-tert-butyl dicarbonate (467 mg, 2.33 mmole) and catalytic amounts of DMAP and heated at reflux for 2 h. The solvent is removed in vacuum and the crude product purified by flash chromatography on silica eluting with dichloromethane:MeOH, 95:5 to afford the protected product as a yellow foam. The compound is dissolved in dichloromethane, cooled to −10° C., treated with MCPBA (285 mg, 1.65 mmol) and stirred at 0° C. for 4 h. The precipitate is filtered, the solvent is evaporated, the crude product further purified by flash chromatography on silica eluting with dichloromethane:MeOH 95:5 to afford the title product as a yellow foam.

Step 5.2: 4-Chloromethyl-N-{4-methyl-3-[4-(1-oxy-pyridin-3-yl)-[2-¹⁴C]pyrimidin-2-ylamino]-phenyl}-benzamide

Hydrochloric acid (4 ml of 4N solution) is added to a suspension of {4-methyl-3-[4-(1-oxy-pyridin-3-yl)-[2-¹⁴C]pyrimidin-2-ylamino]-phenyl}-carbamic acid tert-butyl ester (Step 5.1, 187 mg, 0.48 mmol) in THF (10 ml). The resulting mixture is heated at 70° C. for 1 h. The cooled mixture is evaporated to give a yellow solid which is purified by flash chromatography on silica, eluting with dichloromethane: MeOH 75:35 to afford yellow crystals. The crystals are suspended in THF (10 ml), treated with 25% aqueous ammonium hydroxide (1 drop) and the resulting mixture subjected to ultrasound. The suspension is filtered and the solvent evaporated to afford a yellow foam. This foam is dissolved in THF (10 ml), treated dropwise with a solution of 4-(chloromethyl)-benzoyl chloride (149 mg, 0.8 mmol) in THF (2 ml) and the corresponding mixture is stirred at RT for 2 h. The resulting suspension is centrifuged, the precipitate product washed further with ether, re-centrifuged, the ether removed and the solid dried to afford the title product as brown crystals.

### Example 6

### 4-(4-Methyl-[2,2,3,3,6,6,6,6-D8]piperazin-1-ylmethyl)-N-[4-methyl-3-(4-pyridin-3-yl-pyrimidin-2-ylamino)-phenyl]-benzamide

4-Chloromethyl-N-[4-methyl-3-(4-pyridin-3-yl-pyrimidin-2-ylamino)-phenyl]-benzamide (3.57 g, 8.30 mmol; obtainable in accordance to Example 1 using non-labeled starting materials) is dissolved in dimethyl formamide (50 ml). To this solution is added potassium carbonate (5.74 g, 41.5 mmol) and N-methyl piperazine-2,2,3,3,5,5,6,6-d8 (815 mg, 7.54 mmol, Isotec, Inc. of Miamisburg, Ohio). The mixture is heated to 45-50 ° C. for approximately 12-14 hours. The reaction is monitored by TLC using a solvent system consisting of DCM/EtOAc/MeOH/NH₄OH (25%) (60/10/10/2) with the Rf of the product being approximately 0.30. The DMF is removed in vacuo and the residue is triturated with water. This water wash produces a very gummy residue is dissolved in ethyl acetate, the aqueous phase is concentrated in vacuo, and its residue dissolved in ethyl acetate. The combined material is purified four times using flash chromatography using the same solvent system as described above. The product is placed in a vial, then placed in an Abderhalden drying apparatus under vacuum (0.01 mm of mercury) at a drying temperature of 65° C. (refluxing methanol). MS ES+502.4 (100%) [M+]+, 503.5 (40%), 504.5 (4%); 1H NMR (d₆-DMSO) δ: 2.1 (3H, s), 2.4 (3H, s), 3.6 (2H, s), 7.2 (1H, d), 7.5 (3H, m), 7.8 (2H, d), 8.1 (1H, s), 8.5 (2H, m), 8.8 (1H, s), 9.0 (1H, s), 9.2 (1H, s), 10.1 (1H, s).

### Example 7

### 4-methyl-N-(4-pyridin-3-yl-[1,3-¹⁵N₂, 2,4,5-¹³C₃]pyrimidin-2-yl)-benzene-1,3-diamine

A solution of crude N-(5-amino-2-methyl-phenyl)-[¹³C,¹⁵N₂]guanidine (step 7.7, 157 mg, 0.96 mmol) and 3-dimethylamino-1-pyridin-3-yl-[1,2-¹³C₂]propenone (step 7.3,169 mg, 0.96 mmol) in n-butanol is heated at reflux (130° C.) for 5 hours. Removal of the solvent and purification by flash chromatography eluting with 5:95 pentane:acetone gives pure title product as a yellow solid, ¹H-NMR 400 MHz (CDCl₃) δ: 9.14 (1H, s, ArH), 8.73 (1H, d, J 4, ArH), 8.50 (1H, d octet, J 7,1.2, ArH), 8.37 (1H, m, ArH), 7.70 (1H, s, NH), 7.45 (1H, dd, J 8, 5), 7.17 (1H, dm, J 168, ArH), 7.03-6.99 (2H, m, ArH), 6.46 (1H, dd, J 8, 1.2, ArH), 2.27 (3H, s, CH₃): MS (c+ESI): 283.3 (100%) [M+H]⁺, 284.4 (15%).

Step 7.1: 3-Trimethylstannanyl-pyridine

In a two-necked flask is placed 3-bromopyridine (4 g, 25.3 mmol) followed by diethyl ether (40 ml) under nitrogen. The solution is cooled to −78° C. followed by addition of n-butyl lithium 1.5 M in hexanes (20 ml, 30.4 mmol). The resulting yellow solution is stirred for 10 minutes followed by the addition of a solution of trimethyltin chloride (5.04 g, 25.3 mmol) in diethyl ether (2 ml). The resulting solution is stirred at −78° C. for 20 minutes, then allowed to warm to RT slowly over a period of 1 hour. The solution is partitioned between hexane (50 ml) and water (100 ml), shaken and the organic layer separated. The aqueous phase is extracted a further two times with hexane (50 ml), the organic phases combined and washed with water (100 ml). The hexane phase is dried over sodium sulphate, filtered and the filtrate evaporated to give a crude oil which is passed though a silica gel column eluting with 9:1 hexane:ethyl acetate and the product fractions collected. The fractions are combined, evaporated to give a light yellow oil which is further purified by distillation in a Kugelrohr oven to give the title product which distilled over at 50 mbar,100° C., as a colourless oil.

Step 7.2: 1-pyridin-3-yl-[¹³C₂]ethanone

In a 30 ml ampoule is placed 3-trimethylstannyl pyridine (step 7.1, 1.83 g, 7.56 mmol), freshly distilled [¹³C₂]acetyl chloride (0.67 g, 8.32 mmol), PdCl₂(PPh₃)₂ (0.3 g, 0.44 mmol) and dry benzene (20 ml). The ampoule is sealed and placed in an oil bath at 95° C. overnight.

The ampoule is opened and the contents removed and partitioned between water (30 ml) and ethylacetate (30 ml) and shaken. The organic phase is separated and the aqueous phase re-extracted a further two times with ethyl acetate (2×30 ml). The organic phases are combined and washed with water (30 ml) and dried over sodium sulphate, filtered and the filtrate carefully is evaporated. The crude oil is purified over silica gel eluting with 1:1 hexane:ethyl acetate to give the title product after careful lyophilization.

Step 7.3: (E/Z)-3-Dimethylamino-1-pyridin-3-yl-[1,2-¹³C₂]propenone

In sealed ampoule both 1-pyridin-3-yl-[¹³C₂]ethanone (step 7.2, 500 mg, 4.06 mmol) and N,N-dimethylformamide dimethylacetal (628 mg, 5.28 mmol) are heated to 100° C. overnight. The crude product is purified by chromatography on silica gel eluting with 9:1 dichloro-methane:methanol to give the title product as yellow crystals.

Step 7.4: [¹³C,¹⁵N₂]thiourea-N,N′-dicarboxylic acid tert-butyl ester

To a suspension of sodium hydride (60% dispersion in mineral oil, 1.18 g, 29.61 mmol) in dry THF (50 ml) at 0° C. under argon is added dropwise a solution of [¹³C,¹⁵N₂]thiourea (500 mg, 6.58 mmol) obtained from Aldrich Chem. Co. in dry THF (82 ml). After complete addition, the solution is stirred for 5 minutes followed by the addition of di-tert-butyl dicarbonate (3.16 g, 14.48 mmol) and the corresponding solution is stirred for 1 hour. A saturated solution of NaHCO₃ (13 ml) is added dropwise followed by water (230 ml). The product is extracted into ethyl acetate (3×150 ml), the organic phases combined, washed with brine and dried over sodium sulphate, filtered and the filtrate evaporated to give a crude product as an oil.

Step 7.5: N-(2-Methyl-5-nitro-phenyl)-[¹³C,¹⁵N₂]guanidine-N,N′-acid tert-butyl ester

To a stirred orange solution of crude [¹³C,¹⁵N₂]thiourea-N,N′-dicarboxylic acid tert-butyl ester (step 7.4, 2.35 g, 6.58 mmol), 2-methyl-5-nitroaniline (1.00 g, 6.58 mmol), triethylamine (3.0 ml, 21.71 mmol) in DMF at 0° C. is added solid mercury II chloride (1.97 g, 7.24 mmol). After 15 minutes stirring at RT, ethyl acetate (50 ml) is added and the mixture is filtered through Hyflo (Celite). The ethyl acetate solution is washed with water (50 ml) then brine (50 ml) and dried over sodium sulphate. The drying agent is filtered off and the filtrate evaporated to give an oil which is purified by flash chromatography on silica gel eluting first with 100% hexane then 90:10 hexane:ethyl acetate followed by 80:20 hexane:ethyl acetate to give the title product.

Step 7.6: N-(2-methyl-5-nitro-phenyl)-[¹⁵N₂,¹³C]guanidine

A solution of N-(2-methyl-5-nitro-phenyl)-[¹³C,¹⁵N₂]guanidine-N,N′-acid tert-butyl ester (step 7.5, 445 mg, 1.13 mmol) in 1:1 trifluoroacetic acid (9 ml):dichloromethane (9 ml) is stirred at RT for 1 hour. 2M Sodium hydroxide is added (50 ml) and the yellow solution extracted into ethyl acetate (3×30 ml), combined and washed with water. The organic phase is dried over sodium sulphate, filtered and the filtrate evaporated to afford a yellow oil which is purified by flash chromatography on normal silica gel eluting with a mixture of 90:10:0.5:0.5 dichloromethane:methanol:water:acetic acid to give the title product.

Step 7.7: N-(5-amino-2-methyl-phenyl)-[¹⁵N₂,¹³C]guanidine

A mixture of N-(2-methyl-5-nitro-phenyl)[¹³C,¹⁵N₂]guanidine (step 7.6, 121 mg, 0.62 mmol) in n-butanol (3 ml) containing 10% Pd/C (12 mg) is stirred under 1 atmosphere of hydrogen gas at RT for 16 hours. Additional 12 mg of 10% Pd/C is added and the mixture stirred for a further 16 hours. The mixture is filtered through Hyflo (Celite), washed with a further portion of n-butanol (3×2 ml), combined and the n-butanol removed on a rotary evaporater to give the crude title product.

1. A compound of formula I 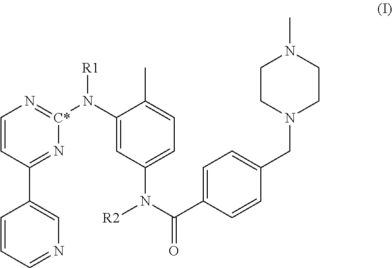
wherein R₁ and R₂ are both hydrogen and C* denotes carbon being labeled by enrichment of one carbon isotope ¹⁴C.

2-13. (canceled)
